(19) United States
(12) Patent Application Publication
(10) Pub. No.: US 2008/0262607 A1
(43) Pub. Date: Oct. 23, 2008

(54) Implant Particularly Stent, and Method For the Production of Such an Implant

(76) Inventor: Martin Fricke, Erfurt (DE)

Correspondence Address: LAW OFFICE OF DELIO & PETERSON, LLC., 121 WHITNEY AVENUE, 3RD FLLOR, NEW HAVEN, CT, 06510, US

(21) Appl. No.: 12/092,913

(22) PCT Filed: Oct. 19, 2006
(86) PCT No.: PCT/EP2006/010105

§ 371 Date: May 7, 2008

(30) Foreign Application Priority Data
Nov. 8, 2005 (DE) 10 2005 053 247.0

(51) Int. Cl. A61F 2/82; A61L 27/28

(52) U.S. Cl. 623/1.46; 427/2.25

(57) ABSTRACT

An implant, particularly a stent, and a method for producing such an implant. The implant (10), particularly the stent (11), has a surface (15) that is coated with a thin layer (16) made of titanium (Ti) and titanium dioxide (TiO₂), the thin layer (16) having an outer surface layer (17) made of the TiO₂ mineral anatase. Particularly preferably, the outer surface layer (17) is formed as a photoactive or photoactivatable, especially photocatalytic or photosensitive layer. The inventive method comprises the following vacuum process steps: plasma pretreatment during which the edges of the base material of the implant are rounded; sputtering of an intermediate layer made of titanium (Ti); sputtering of a surface layer made of the titanium dioxide (TiO₂) mineral anatase.

The invention relates to an implant, particularly a stent, the surface of which is coated with a thin layer, as well as to a method for producing such an implant, particularly a stent, according to the preamble of patent claim 10.

Disclosed in EP 1 535 660 A1 is a device for producing at least one fluid reaction product from at least one fluid starting substance by means of chemical reaction in the plasma of dielectrically impeded discharges. This device has a first electrode made of a porous, electrically conducting body, a second electrode made of a suchlike body, and a dielectric layer provided between the flow-through electrodes. The dielectric layer is a thin layer, preferably a photocatalytic thin layer made of titanium dioxide (TiO₂), especially preferably made of the TiO₂ mineral anatase. Between its porous body and the dielectric layer of this body, each of the mentioned electrodes has a transition layer preferably made of titanium (Ti) as adhesion mediator. According to the aforementioned document, this device is suitable, in the case of motor vehicles operated with fuel cells, to produce large amounts of hydrogen from hydrocarbons, particularly from methane or natural gas, liquefied gases, gasified gasoline or gasified diesel, it being possible for such a process to take place on-board, that is, directly in the motor vehicle. The device described here is also suitable for producing, in particular, hydrogen-rich fuel gas for motor vehicles equipped with fuel cells.

Disclosed in DE 102 10 465 A1 is a photocatalytic element for cleaving hydrogen-containing compounds and a method for producing such a photocatalytic element. For the photocatalytic element, a photocatalytically active binder-free thin layer made of a photosemiconductive material is formed on a support and the support has an open-pored structure or forms such an open-pored structure. In the aforementioned method, a photocatalytically active, binder-free thin layer is formed on a support having an open-pored structure by means of a plasma-based vacuum coating method. In this document, too, special reference is made to the operation of fuel cells. Further mentioned is the use of the mentioned photocatalytic elements for deodorizing or disinfecting, for example, exhaust gas from industrial or agricultural processes or the employment of them for cleaning water contaminated by organohalogen compounds or for eliminating the carcinogenic or mutagenic effects of such compounds. Furthermore, according to this document, the photocatalytic thin layer made of titanium dioxide of anatase modification is formed by reactive pulse magnetron sputtering. In doing so, an intermediate layer can consist of high-purity titanium, which is formed, for example, by means of a sputtering process.

Disclosed in DE 601 06 962 T2 is a porous, metallic stent coated with a ceramic layer and furnished with a pharmacologically active substance, the pores of the stent being capable of taking up pharmacologically active substances and of eluting them. The ceramic layer can consist of titanium dioxide (TiO₂). This document discloses a method for producing a polymer coating or ceramic coating of the porous metallic stent by using processes of spotted film deposition and accordingly a marked modification of the surface of a metallic stent so as to make possible the continuous delivery of medications in different intensity from the stent.

Furthermore, disclosed in DE 102 43 132 A1 is a method for producing a biocompatible metal-ion-containing titanium oxide coating on an implant, the metal ions being elutable under physiological conditions and being distributed homogeneously in the coating. Further disclosed in this document is a method for producing such an implant. Titanium oxide is understood here to mean essentially titanium dioxide. Created through the method described here is a titanium oxide coating or an implant having a titanium oxide coating, it being possible for the metal ions to dissolve out with antimicrobial effect under physiological conditions. After a certain period of time, once the antimicrobially active metal ions have largely dissolved out, the antimicrobial effect of the coating declines and the implant is integrated into the body tissue and hence is biocompatible.

So-called percutaneous transluminal angioplasty (PTA) of blood vessels, in particular of coronary arteries, serves to eliminate narrowings or so-called stenoses, which impede the blood supply of, for example, human organs. An excessive proliferation of inner vessel wall, referred to as the vessel intima, within the stent is regarded as the primary cause of a restenosis, that is, a renewed narrowing of the blood vessel in question.

The invention is based on the problem of creating an implant, particularly a stent, the compatibility of which with body tissue is improved in an especially simple manner, thereby preventing a restenosis. The invention is further based on the problem of presenting a method for producing such an implant, particularly a stent.

This problem is solved by an implant having the features of patent claim 1 and by a method having the features of patent claim 10. Advantageous further developments are the subject of the respective dependent claims.

In accordance with the invention, the implant, particularly the stent, is coated with a thin layer made of titanium (Ti) and titanium dioxide (TiO₂), the thin layer having an outer surface layer made of the TiO₂ mineral anatase. An implant with such a thin layer exhibits a good biocompatibility and a likewise good long-term compatibility in association with body tissue. Besides improved sliding properties and an outstanding, secure adhesion of the thin layer to metallic and also to nonmetallic base materials of the implant, also referred to as support materials, the inventive implant exhibits a good long-term tolerance and a good ingrowth in tissue in the case of stents in the vessel wall. Thus, in accordance with the invention, an improved compatibility of the implant is achieved not through pharmacologically active substances or through antimicrobially active metal ions that dissolve out, as in the prior art mentioned, but solely through the physical structure of the thin layer and the advantages ensuing from it.

In accordance with an especially preferred embodiment of the invention, the outer surface layer is formed as a photoactive or photoactivatable, particularly photocatalytic or photosensitive, surface layer. The photoexcitation of the outer surface layer makes it superhydrophilic, thereby improving its sliding properties and preventing deposits, such as, for example, in the case of a thrombosis. Furthermore, the photoexcitation of the outer surface results, via photocatalytic processes at the interface of the photosensitive layer and the intima tissue of substances that prevent restenosis or cause it to regress. The so-called photoactivatable superhydrophilicity also improves the sliding properties during angioplasty and diminishes the risk of acute thrombosis. On account of the superhydrophilicity, deposits at the implant base material are also prevented.

In accordance with another further development of the invention, the thin layer has an intermediate layer, made of pure titanium (Ti), which is deposited on the surface of the base material of the implant and serves as a connection between the base material of the implant and the outer surface layer. The intermediate layer is accordingly a kind of joining layer, which joins the outer surface layer made of the TiO₂ mineral anatase firmly to the base material of the implant, so that the outer surface layer represents a fixed or immobilized surface layer. However, this intermediate layer made of pure titanium is not only a joining and intermediate layer, but also acts as a so-called barrier layer for the metal ions (if present) of the base material of the implant. Thus, these metal ions cannot reach the outside and therefore cannot enter body tissue or the blood circulation as in the prior art mentioned. The intermediate layer made of titanium has ductile properties, whereas the outer surface layer made of the mentioned TiO₂ mineral has a ceramic and monocrystalline structure.

In accordance with another preferred further development of the invention, the edges of the base material of the implant are rounded. In this way, the inner vessel wall experiences as little irritation as possible when the stent is inserted and it is thereby possible to reduce the risk of restenosis. A renewed narrowing or occlusion of the vessel is thereby largely minimized. An implant designed in this manner in accordance with the invention can therefore be inserted especially gently at the desired site in, for example, a blood vessel. On account of the rounded edges of the base material, moreover, the sliding properties of the implant are improved in the blood vessel, for example, and the danger of lesions of the vessel wall during a PTA are reduced.

Advantageously, the thickness of the intermediate layer is about 200 to 1000 nm and preferably that of the outer surface layer is about 100 to 1000 nm. In particular, the individual layers and accordingly the thin layer can be kept extremely thin overall.

In accordance with another further development of the invention, the intermediate layer and the outer surface layer are each an all-sided plasma coating of the implant. It follows from this that the base material of the implant, particularly the stent, is entirely surface-coated, that is, also at the inner-lying surfaces of the implant, for example. In this way, the inventive implant has a good biocompatibility overall and not only at individual sites, making it possible to markedly reduce the risk of restenosis.

In accordance with another further development of the invention, the thin layer is deposited on the entire surface of the deflated implant, preferably the stent, as a closed surface layer. It follows from this that, when the thin layer is deposited, the implant is preferably in the non-expanded or non-unfolded state.

According to another further development of the invention, the outer surface layer can be preferably photodynamically activated or reactivated by illumination with blue light or UVA light in the wavelength range between 360 and 460 nm, it being possible for an activation or an in vivo reactivation of the outer surface layer to take place by means of an optical fiber, which preferably has fiber optics that radiate light in the radial direction. In this respect, the photoactivation of the outer surface layer can take place immediately prior to the angioplasty by photoactivation of the hydrophilicity by means of a simple illumination or photoexcitation of the entire implant surface. It is further possible to reactivate the hydrophilicity or the special layer properties once again for post-treatment at a later point in time, it being possible for such a reactivation to be the mentioned in vivo reactivation. In so doing, optical fiber cables and microfiber optics inserted into the implant, particularly the stent, can be employed for carrying out the photochemical processes at the interfaces of the inner side of the implant, particularly the stent, by using catheters. In this respect, this further development makes it possible to diminish the risk of restenosis by preventing ingrowth processes by using the mentioned photocatalysis in the framework of an in vivo reactivation. The mentioned optical fibers with their fiber optics therefore makes possible an irradiation of the implant at its site of use, that is, for example, in a blood vessel, such as a coronary artery.

The inventive method for producing the implant, particularly the stent, comprises the following vacuum process steps: plasma pretreatment with rounding of the edges of the implant base material; sputtering of an intermediate layer made of titanium (Ti); sputtering of a surface layer made of the titanium dioxide (TiO₂) mineral anatase. Accordingly, the rounding of the edges of the implant base material can take place in time prior to the application of the thin layer, so that the implant is furnished exclusively in rounded edges with the inventive thin layer.

Advantageously, the plasma pretreatment comprises a plasma surface cleaning and a plasma polishing. Accordingly, exclusively a highly clean, well-biocompatible implant is furnished ultimately with the special thin layer consisting of two surface layers.

According to another further development of the invention, the intermediate layer made of titanium and the surface layer made of the TiO₂ mineral anatase are deposited by way of reactive pulse magnetron sputtering (PMS). This application of the two layers thus takes place on the entire outer surface of the mechanically finished implant, such as a raw stent. Accordingly, the sputtering process also includes the inner-lying outer surfaces of the implant.

Exemplary embodiments of the subject of the invention will be described in greater detail below on the basis of the drawing, all described and/or graphically illustrated features constituting, in themselves or in any combination, the subject of the present invention, regardless of their summary in the claims or referral back to them. Shown are:

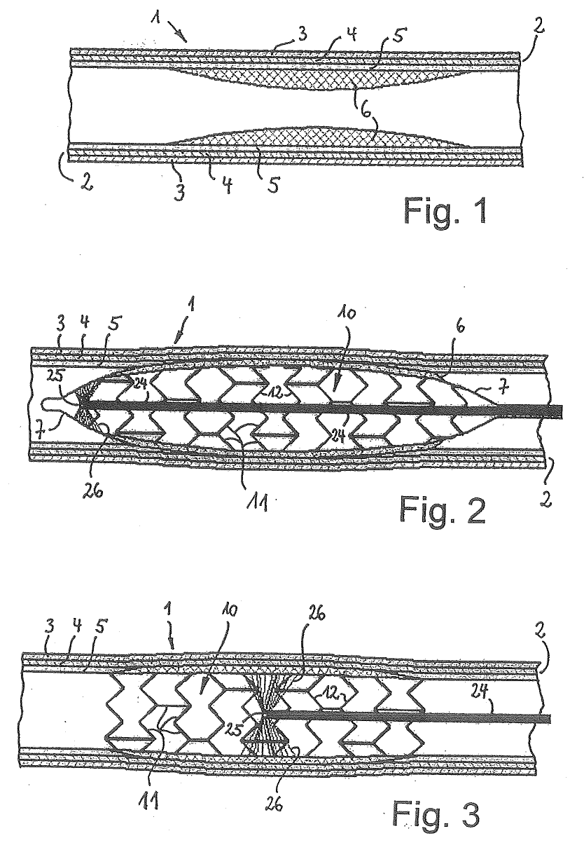

FIG. 1 a schematic lengthwise section through a narrowed vessel, such as, for example, a coronary artery;

FIG. 2 a schematic, partially lengthwise section through a narrowed vessel with an inserted balloon catheter and mounted implant in the form of a stent;

FIG. 3 a schematic, partially lengthwise section through a balloon-dilated vessel after an expansion of the implant and removal of the balloon catheter;

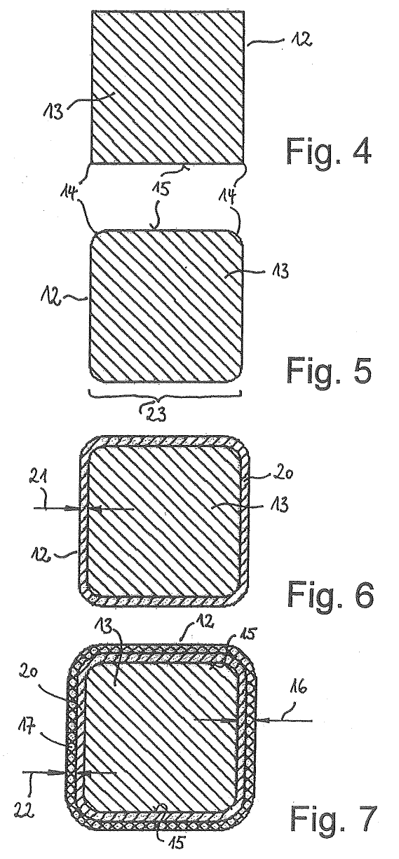

FIG. 4 a schematic cross section through a strut of the implant;

FIG. 5 a schematic cross section through the strut after rounding of the edges;

FIG. 6 a schematic cross section through the strut after deposition of an intermediate layer made of titanium on the strut according to FIG. 5;

FIG. 7 a schematic cross section through the strut after deposition of a surface layer made of the titanium dioxide mineral anatase on the strut according to FIG. 6;

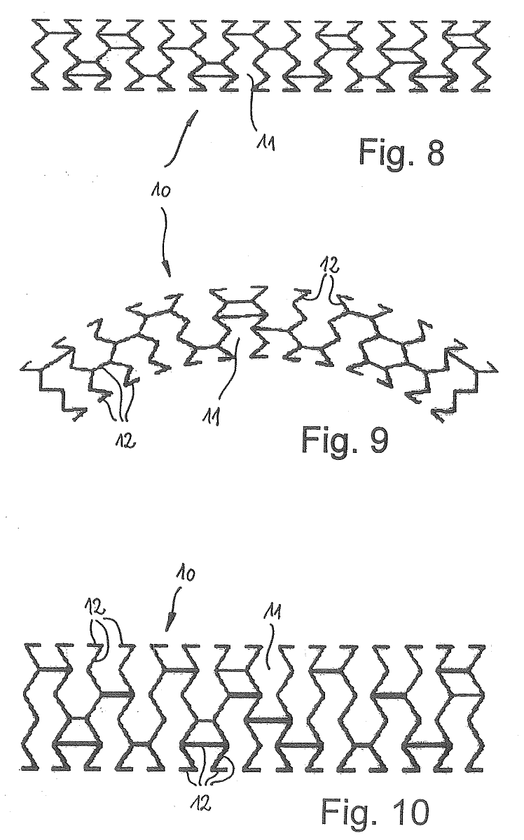

FIG. 8 an exemplary, schematic illustration of an implant in the deflated state;

FIG. 9 an exemplary, schematic illustration of an implant in the deflated, bent state; and

FIG. 10 an exemplary, schematic illustration of an implant in the inflated, that is, expanded or unfolded, state.

Shown in FIG. 1 is a schematic lengthwise section through a vessel 1—for example, a coronary artery. The vessel 1 has a vessel wall 2, which is formed from three layers. These are, from radially outward to radially inward, the outer vessel wall layer 3, referred to as the tunica externa, the middle vessel wall layer 4, referred to as the tunica media, and the inner vessel wall layer 5, referred to as the tunica intima.

The vessel 1 shows a stenosis 6 due to plaque. It is clear that the aforementioned structure of the vessel 1 is merely an exemplary description of such a vessel. Vessels having a different structure could be included equally well.

Illustrated in FIG. 2 is a schematic lengthwise section through the narrowed vessel 1 with an inserted balloon catheter 7 and a mounted implant 10, namely, a stent 11.

The implant is formed as a stent merely by way of example. In this respect, the invention is not limited to an implant formed as a stent, but rather the invention includes other types of implants as well.

Furthermore, the invention also includes medical instruments, particularly dental instruments, the surfaces of which are formed at least partially in the manner in accordance with the invention. In this respect, the invention relates generally to implants as well as medical instruments.

In FIG. 2, the stent 11 is illustrated partially inflated, that is, unfolded.

In contrast, FIG. 3 shows a schematic, partially lengthwise section through the vessel 1 with a now fully inflated stent and removed balloon catheter 7.

FIG. 2 and FIG. 3 will be addressed in more detail later.

In the following, the inventive implant 10 will be described more exactly with reference to FIG. 4 to 7.

As already mentioned, the implant 10 is formed preferably, but not exclusively, as a stent 11. Such a stent 11 has numerous arms 12, also referred to as struts, of which one is illustrated in cross section in each of FIGS. 4 to 7. Illustrated schematically in FIG. 4 is a cross section through such a strut of the stent 11. The strut is fabricated, for example, of surgical steel, such as, for example, surgical steel 316L, from Nitinol®, a corrosion- and shock-resistant, steel-hard titanium-nickel alloy. However, the base material 13 of the stent 11 can also consist of another metal, of other alloys, or else of nonmetals and plastics. For example, the cross section through a strut 12 shown in FIG. 4 results from a tubular sleeve after laser cutout of the material.

In the illustration according to FIG. 5, the edges 14 of the base material 13 of the strut 12 of the stent 11 are rounded, which will be discussed in greater detail later.

In accordance with the invention, the implant 10, that is, for example, the stent 11, has a surface 15 that is coated with a thin layer 16 made of titanium (Ti) and titanium dioxide (TiO₂), the thin layer 16 having an (outer) surface layer 17 made of the TiO₂ mineral anatase. This surface layer 17 is preferably formed as a photoactive or photoactivatable, particularly photocatalytic or photosensitive surface layer.

The invention relates, in particular, to a photoactivatable thin layer surface made of the TiO₂ mineral anatase in the form of a monocrystalline layer as bioactive surface of an implant and/or a medical instrument.

The thin layer 16 further has an intermediate layer 20, which is deposited on the surface 15 of the base material 13 of the implant 10 and is made of pure titanium (Ti) as connection between the base material 13 of the implant 10 and the surface layer 17. As already mentioned, the two described layers 17, 20 of the thin layer 16 are deposited on the rounded base material 13 of the implant 10. It follows from this that, prior to the deposition of the thin layer 16, the edges 14 or the corners of the base material are initially rounded in a preceding processing step.

It is noted that the intermediate layer made of titanium can be dispensed with insofar as the base material of the implant or of the medical instrument is fabricated from titanium or at least contains titanium to a notable extent.

The thickness 21 of the intermediate layer 20 is about 200 to 1000 nm and preferably the thickness 22 of the outer surface layer 17 is about 100 to 1000 nm.

As indicated merely schematically in FIG. 4 to 7, the intermediate layer and the outer surface layer 20, 17 are each an all-sided coating, preferably an all-sided plasma coating, of the implant 10. According to a preferred embodiment of the invention, the thin layer 16 is deposited as a closed layer 17, 20 on the entire surface 15 of the deflated implant 10, preferably the stent 11.

The edge length 23 of the base material 13 is preferably about 0.1 mm. It is clear that the surface 15, different from what is shown in FIG. 4 to 7, can also have a curved shape.

According to an especially preferred embodiment of the invention, the outer surface layer 17 can be photodynamically activated or reactivated by illumination with blue light or UVA light in the wavelength range between 360 and 460 nm. Such an activation or reactivation of the outer surface layer 17 can take place, according to the embodiment of the invention shown in FIGS. 2 and 3, by means of an optical fiber 24. As indicated in FIGS. 2 and 3, the fiber optics 25 are designed in such a manner that the light 26 leaves the optical fiber 24 roughly in the radial direction. As further shown in FIGS. 2 and 3, the reactivation of the outer surface layer 17 can preferably be an in vivo reactivation.

In the following, the inventive method will be described in greater detail for the production of an implant, particularly a stent.

The inventive method comprises the following vacuum process steps:
plasma pretreatment with rounding of the edges of the implant base material;
sputtering of an intermediate layer made of titanium (Ti);
sputtering of a surface layer made of the titanium dioxide (TiO₂) mineral anatase.

According to a preferred further development of the invention, the plasma pretreatment comprises a plasma surface cleaning and a plasma polishing.

In this respect, the cross section of a strut 12 of the implant 10, shown in FIG. 4, illustrates the base material 13 in raw form, whereas the cross section according to FIG. 5 has already undergone the mentioned plasma pretreatment, particularly a rounding of the edges, unevenness, and processing burrs of the implant base material.

The layers 20, 17, made of titanium and the TiO₂ mineral anatase, are deposited by reactive pulse magnetron sputtering (PMS). The plasma pretreatment can also include the polishing or the smoothing out of unevenness on the surface 15 of the base material 13.

As already indicated, the stent 11 in FIG. 2 is present in a partially inflated, that is, expanded state. According to this illustration, the stent 11 here is still mounted on the balloon catheter 7. The stenosis 6 here is illustrated as already being pressed radially outward in comparison to the illustration according to FIG. 1, it being thereby possible to effect a slight dilation of the vessel 1, as indicated in FIGS. 2 and 3.

The vessel according to FIG. 3 is balloon-dilated and in a state following an expansion of the stent 11 and removal of the balloon catheter 7. Accordingly, the stent 11 is fully inflated, that is, unfolded. According to FIG. 2, the optical fiber 24 together with its fiber optics 25 are situated inside of the balloon catheter 7. By means of the optical fiber and fiber optics, the activation/reactivation, that is, the carrying out of a photodynamic process, at the photoactive outer surface layer 17 is carried out by application of light. Shown in FIG. 3 is the optical fiber 24 together with its fiber optics 25 without the balloon catheter 7. In its inflated state, the stent is thus capable of completely eliminating the cross-sectional narrowing, as indicated in FIG. 1, caused by the stenosis 6 (see illustration according to FIG. 3).

Shown in FIG. 8 is the implant 10 in the form, by way of example, of a stent 11 in a deflated, that is, collapsed or folded, state. According to FIG. 9, a stent deflated as in FIG. 8 has an arched shape, whereas, in FIG. 10, a stent 11 is shown in the inflated, that is, expanded or unfolded state. In the state shown in FIG. 10, for instance, the stent 11 is situated in the vessel 1 according to FIG. 3. These illustrations highlight the facile mobility, such as bendability and good flexibility, of the stent.

It is clear that the implant 10 or the stent 11 can assume numerous different shapes and, in this respect, can be formed in a large number of embodiments. The stents are illustrated merely by way of example in FIG. 8 to 10.

In this respect, the use of light for in vivo activation or reactivation of implant surfaces represents a contribution to minimally invasive medicine. As previously mentioned, it is possible to insert the inventive implant with an already photocatalytically excited outer surface layer into the blood vessel 1 and to carry out the operation of renewed activation or reactivation of the outer surface layer in the placement state of the stent in the blood vessel a second time.

The stent 11 is, for example, an implantable vessel support made of wire mesh or tiny metal tubes with recesses in the tube wall for treatment of occlusions and narrowings in vessels. As previously mentioned, it is possible by means of the invention to further minimize the acute risks during the PTA and, in particular, to reduce the risk of restenosis following a PTA, this taking place through the mentioned photoactivation or in vivo reactivation of the photoactive interfaces of the surface layer of the struts. Accordingly, the reactivation can be carried out at photoactivated interfaces of the surface layer of struts with the intima and, if appropriate, the newly formed, proliferating neointima in order to suppress the mentioned restenosis or at least to strongly limit it. The inflation, that is, the unfolding, of the stent can take place in vivo through the mentioned balloon mounting or by self-expansion.

The intermediate layer 20, made of pure titanium, has the function of adapting the coefficients of expansion of the base material 13, also referred to as support material, the stent struts, and the photoactive outer surface layer made of the mentioned TiO₂ mineral anatase and of joining the latter-mentioned anatase layer to the base material in a tightly adhering manner. The intermediate layer 20, made of pure titanium, has the further function of accommodating the forces or stresses, such as, for example, tensile stresses, compression stresses, and torsional stresses, that arise during the plastic deformation of the struts of the stent as a result of the inflation, that is, unfolding or expansion, of preventing ablations, and of impeding corrosions during the formation of microcracks in the layer as well as of making possible the growth (recrystallization) of new anatase nanocrystallites in the microcracks so as to heal the layer surface. The mentioned TiO₂ mineral anatase exists in a layer made up of monocrystallites of the anatase morphology of TiO₂ as photoactivatable surface.

The mentioned materials Ti and TiO₂ are hemo- and histocompatible materials and have undergone long-term testing as implant materials.

In this respect, the inventive implant represents an alternative to drug-delivering implants, particularly stents, and, in a simple and low-cost variant, offer the possibility of preventing a restenosis.

Accordingly created is an implant or, in general, a medical article, such as, for example, a medical instrument, the compatibility of which, especially the biocompatibility of which, is improved in a simple manner and which is capable of largely preventing, in particular, a restenosis. In addition, a method for producing such an implant or medical article is presented.

1-12. (canceled)

13. An implant having a base material with a surface including a film, said film comprising titanium (Ti) and titanium dioxide (TiO2), wherein an outside layer of said film includes TiO2-Mineral Anatase.

14. The implant of claim 13 wherein said outside layer includes a photoactive or photo-activatable material.

15. The implant of claim 13 wherein said outside layer includes a photo catalytic or photo-sensitive material.

16. The implant of claim 13 wherein said film includes an intermediate layer deposited on said base material surface, said intermediate layer including pure titanium (Ti), and situated between said base material of the implant and said outside layer.

17. The implant of claim 13 wherein said base material includes rounded edges.

18. The implant of claim 13 including said intermediate layer having a thickness of about 100 to 1000 nm.

19. The implant of claim 13 including said intermediate layer having a thickness of about 200 to 1000 nm.

20. The implant of claim 13 further including a plasma coating on said film.

21. The implant of claim 20 including having said plasma coating on said intermediate layer.

22. The implant of claim 20 including having said plasma coating on said outside layer.

23. The implant of claim 13 wherein said film is deposited as a closed layer on the entire surface of said implant.

24. The implant of claim 13 wherein said implant comprises a stent.

25. The implant of claim 13 including having said outside layer photo-dynamically activated or re-activated when illuminated with blue light or UVA light in the wavelength range between 360 nm and 460 nm.

26. The implant of claim 25 including having said photo-dynamically activated or re-activated outside layer illuminated as an in-vivo-reactivation, said outside layer sensitive to illumination by an optical fiber exhibiting fiber optics radiating light in radial direction.

27. A method for manufacturing an implant, said method comprising the following process steps:
rounding edges of a base material of said implant;
pretreating said base material with a plasma coating; and
forming a film on said base material, including:
sputtering an intermediate layer of titanium (Ti) on said base material; and
sputtering an outside layer of titanium dioxide (TiO2) mineral anatase on said intermediate layer.

28. The method of claim 27 wherein said base material comprises surgical steel.

29. The method of claim 27 including having said plasma pretreatment comprise a plasma surface cleaning and a plasma polish.

30. The method of claim 27 including having said intermediate layer and said outside layer pulsed by a reactive pulse sputtering magnetron process.
